(19) United States
(12) Patent Application Publication
(10) Pub. No.: US 2011/0090222 A1
(43) Pub. Date: Apr. 21, 2011

(54) VISUALIZATION OF SCARING ON CARDIAC SURFACE

(75) Inventors: Julian Ibarz, Plainsboro, NJ (US); Liron Yatziv, Fremont, CA (US); Romain Moreau-Gobard, Palo Alto, CA (US); James Williams, Nurnberg (DE)

(73) Assignee: Siemens Corporation, Iselin, NJ (US)

(21) Appl. No.: 12/897,974

(22) Filed: Oct. 5, 2010

Related U.S. Application Data
(60) Provisional application No. 61/251,887, filed on Oct. 15, 2009.

(51) Int. Cl. G06T 17/00; A61B 6/03; G06T 15/00; G06T 15/40; G06T 15/50

(52) U.S. Cl. 345/422; 600/425; 345/419; 345/426; 345/424

(57) ABSTRACT

A method for imaging a myocardial surface includes receiving an image volume. A myocardial surface is segmented within the received image volume. A polygon mesh of the segmented myocardial surface is extracted. A surface texture is calculated from voxel information taken along a path normal to the surface of the myocardium. A view of the myocardial surface is rendered using the calculated surface texture.

### CROSS-REFERENCE TO RELATED APPLICATION

The present application is based on provisional application Ser. No. 61/251,887, filed Oct. 15, 2009, the entire contents of which are herein incorporated by reference.

### BACKGROUND OF THE INVENTION

1. Technical Field

The present disclosure relates to visualization of cardiac scars and, more specifically, to visualization of scaring on cardiac surface.

2. Discussion of Related Art

Myocardial scarring is the establishment of fibrous tissue that replaces normal tissue destroyed by injury or disease within the muscular tissue of the heart. Myocardial scarring often occurs as a result of myocardial infarction but may also result from surgical repair of congenital heart disease. This scarring may result in a disruption to the electrical conduction system of the heart, and may also affect surrounding heart muscle tissue.

As such disruptions to the electrical conduction system of the heart may contribute to cardiac dysrhythmia and other problems, effective visualization of cardiac scarring may be useful in performing various interventions such as radio frequency ablation, which may be used to treat dysrhythmia and other problems.

For example, during cardiac visualization, cardiac scars may become more visible as contrast agent is absorbed in the scar tissue. Accordingly, complaining cardiac image volumes acquired before and after the contrast agent is absorbed in the scar tissue is a common way to visualize scars. However, it may be difficult to adequately visualize the scars with regular volume rendering techniques.

### SUMMARY

A method for imaging a myocardial surface includes receiving an image volume. A myocardial surface is segmented within the received image volume. A polygon mesh of the segmented myocardial surface is extracted. A surface texture is calculated from voxel information taken along a path normal to the surface of the myocardium. A view of the myocardial surface is rendered. The rendering includes imposing the calculated surface texture onto the polygon mesh.

The image volume may be received from an image database, a computed tomography (CT) scanner, or a C-arm CT scanner. Segmentation of the myocardial surface may include loading a pre-determined segmentation or calculating segmentation by applying a detection algorithm to the image volume. Extracting a polygon mesh may be performed by applying a marching squares approach to the segmented myocardial surface. Rendering the view of the myocardial surface may include rendering the polygon mesh in a depth buffer of a graphical processing unit (GPU) using a rasterization algorithm. Position information of the visible myocardial surface may be extracted from the rendering of the myocardial surface in the depth buffer rather than the image volume. Calculating the surface texture from the voxel information taken along a path normal to the surface of the myocardium may be performed starting from the previous extracted position information. The path normal to the surface of the myocardium ay be a smoothed normal.

Regions of scarring may be highlighted on the rendering of the myocardial surface. Highlighting of scarring may include calculating a derivative of the rendered surface. Highlighting of scarring may include application of a Sobel filter to the highlighted regions of scarring.

Scarring may be automatically segmented from the rendered surface mesh. Scarring may be automatically segmented from the rendered surface mesh based on the highlighting.

A user may be allowed to change one or more parameters of display or segmentation and then re-rendering the view of the myocardial surface in real-time based on the changed parameters.

A method for applying texture to a polygon mesh includes casting a ray from a point of view. The ray intercepts a three-dimensional structure within an image volume. A direction normal to the surface of the three-dimensional structure is determined at the point at which the ray intercepts the surface of the structure. A set of voxels of the three-dimensional structure is analyzed along the normal direction including ascertaining voxel color and transparency. As the normals may be smoothed before this point, the set of voxels of the three-dimensional structure may be analyzed along the smoothed normal direction. The set of voxels is combined based on the ascertained color and transparency to create a texture element. The created texture element is applied to the polygon mesh.

Prior to determining the direction normal to the surface of the structure, normals of the surface of the structure may be smoothed. The three-dimensional structure may include a myocardium and the polygon mesh may be a representation of a surface of the myocardium.

A computer system includes a processor and a non-transitory, tangible, program storage medium, readable by the computer system, embodying a program of instructions executable by the processor to perform method steps for imaging a myocardial surface. The method includes receiving an image volume. A myocardial surface is segmented from within the received image volume. A polygon mesh of the segmented myocardial surface is extracted. A surface texture is calculated from voxel information taken along a path normal to the surface of the myocardium. A view of the myocardial surface is rendered. The rendering includes imposing, on-the-fly, the calculated surface texture onto the polygon mesh without having to do a texture mapping. Scarring is highlighted on the rendering of the myocardial surface. The scarring is segmented on the rendering of the myocardial surface based on the highlighting.

Rendering the view of the myocardial surface may include rendering the polygon mesh in a depth buffer of a graphical processing unit (GPU) using a rasterization algorithm. Position information of the visible myocardial surface may be extracted from the rendering of the myocardial surface in the depth buffer rather than the image volume. Calculating the surface texture from the voxel information taken along a path “normal” to the surface of the myocardium may be performed starting from the previous extracted position information. The path normal to the surface of the myocardium may be a smoothed normal.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a flow chart illustrating an approach for cardiac scar visualization according to an exemplary embodiment of the present invention;

FIG. 2 is a diagram illustrating a traditional approach for texture mapping;

FIG. 3 is a diagram illustrating an approach for applying a texture according to an exemplary embodiment of the present invention;

FIG. 4 is an example of a real-time segmentation result 20 that uses difference between max and mean along the ray to visualize cardiac surface scarring according to an exemplary embodiment of the present invention;

FIG. 5 is an example of a real-time segmentation result 30 including scar highlighting using derivative of the segmented image according to an exemplary embodiment of the present invention; and

FIG. 6 shows an example of a computer system capable of implementing the method and apparatus according to embodiments of the present disclosure.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention seek to provide methods for visualization of cardiac scars that may be located inside the myocardium from within image volumes such as those acquired by computed tomography (CT) or C-Arm CT. These methods may use parallel computing and may be efficiently implemented within a graphic processing unit (GPU). In so doing, a user may be able to change in real-time the visualization parameters to better highlight the scars.

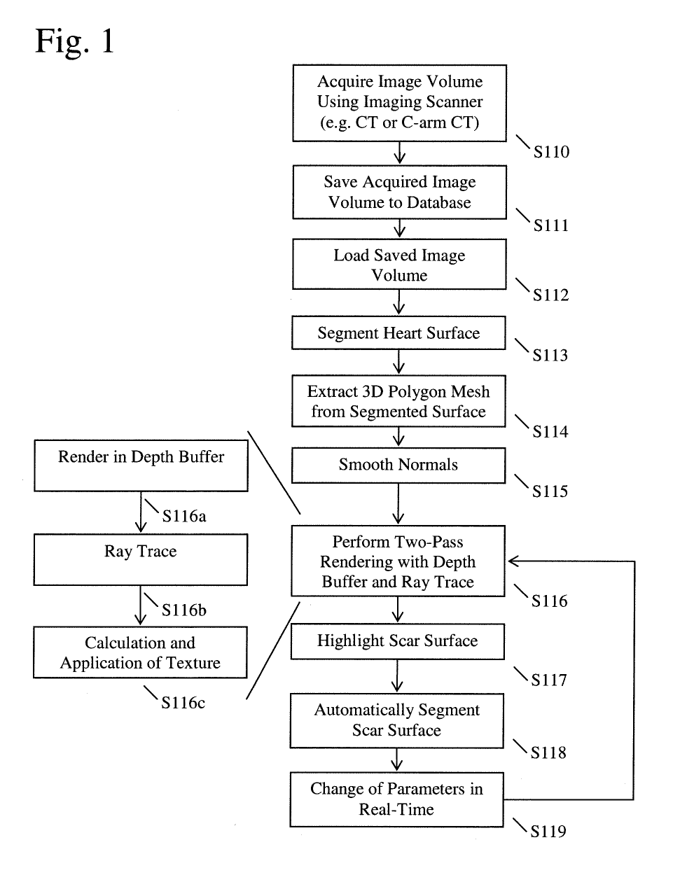

FIG. 1 is a flow chart illustrating an approach for cardiac scar visualization according to an exemplary embodiment of the present invention. Various techniques according to exemplary embodiments of the present invention may begin with the loading of an image volume (Step S112). The image volume may either be retrieved from a digital storage space such as a patient record database or acquired directly from a three-dimensional medical imaging scanner. The image volume may have been acquired from a three-dimensional medical imaging scanner such as a CT or C-arm C (Step S110) and then saved to the digital storage space (Step S111) prior to the loading of the image volume (Step S112).

The surface of the heart may then be segmented (Step S113). Segmentation of the surface of the heart may be defined as determining which of the voxels of the image volume represent the outer surface of the heart. Segmenting of the surface of the heart may include either loading a pre-determined segmentation or calculating segmentation by applying an algorithm for detecting the surface of the heart. After segmentation, a polygon mesh may be extracted from the segmented surface of the heart (Step S114). An example of a suitable mesh extraction technique is the marching cubes algorithm, however, other known techniques for polygon mesh extraction may be used to generate a polygon mesh that represents the surface of the heart.

Extraction of the surface mesh may result in a three-dimensional polygon mesh representing the heart surface. Next, the three-dimensional mesh may be rendered for viewing. Rendering of the three-dimensional mesh may include applying a surface texture over the surface mesh so that a two-dimensional rendering of the surface of the heart, complete with surface texture, may be displayed for a user. In order to determine an appropriate surface texture, the image volume may be consulted. The surface texture may then be determined by identifying color values and transparency values of the corresponding portion of the image volume.

Traditionally, in determining a texture to be applied, the color values and the transparency values may be assessed along a ray that is traced from a viewpoint. Ray casting may be performed starting from the surface of the myocardium. To do so, rasterization may then be used to find the intersection of a pixel ray and the myocardium surface. Then the surface texture in that region may be determined by combining the colors of the voxels that intercept the corresponding ray while accounting for their degree of transparency so that a realistic surface texture may be created.

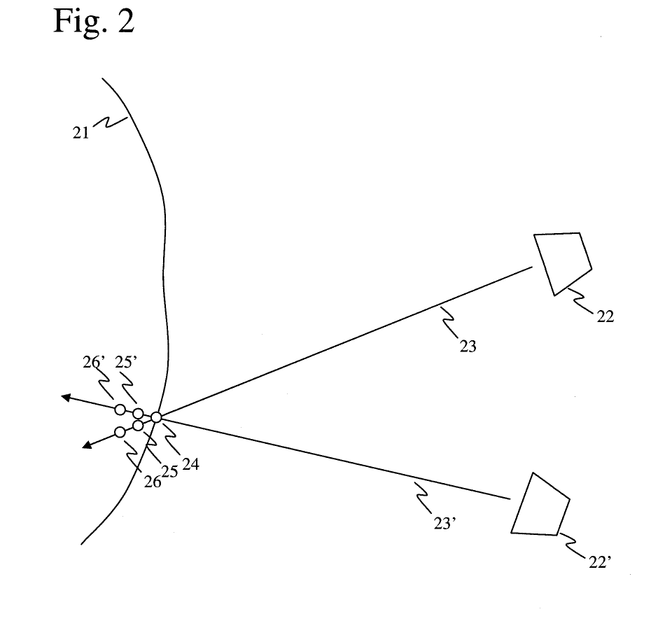

FIG. 2 is a diagram illustrating a traditional approach for performing classical volume rendering using a ray casting technique. Here, the surface texture for the polygon mesh is determined by analyzing the image volume 21 along a ray 23 that has been cast from a point of view 22. The ray 23 intercepts the image volume 21 at various voxels, for example, voxels 24, 25, and 26. At each intercepted voxel, color and transparency is analyzed to produce a texture element (texel) that corresponds to a location on the surface of the polygon mesh. Texels are later imposed on corresponding polygons of the mesh to give the mesh, which would otherwise appear as a wire-frame, an accurate appearance. It should be noted that each of the voxels 24, 25, and 26 lay along the path of the ray 23. While this approach may provide an accurate representation of the appearance of the surface of the image volume, a problem may be encountered as the camera angle is changed, for example, to a second point of view 22′. As the camera angle changes from the first point of view 22 to the second point of view 22′, a second ray 23′ is traced. The second ray also intercepts the surface of the image volume at voxel 24. Accordingly, the previously calculated texel must now be recalculated for the same corresponding mesh polygon. This time, the texel is calculated by utilizing color and transparency information for voxels 24, 25′, and 26′, where voxels 25′ and 26′ are different than voxels 25 and 36.

Exemplary embodiments of the present invention may continuously recalculate polygon mesh surface shading. Rather than relying on texture mapping in the classical approach where a 2D texture image is computed and then imposed upon a 3D object surface using 2D texture coordinates, surface color is computed on-the-fly. As this approach may be computationally more expensive than classic texture mapping approaches, exemplary embodiments of the present invention may achieve acceptable speed by utilizing a graphical processing unit (GPU) for the on-the-fly computation of surface color. This approach may avoid creation of artifacts in surface shading which may be commonly found when using texture mapping.

Moreover, according to classical volume rendering techniques such as that described above where texture is computed along the ray cast from the point of view to the 3D structure, texels are generally calculated once and is not intended to change as the point of view moves. As described above, this may present problems when applied to myocardial surface visualization as texture will tend to be different depending on the current point of view.

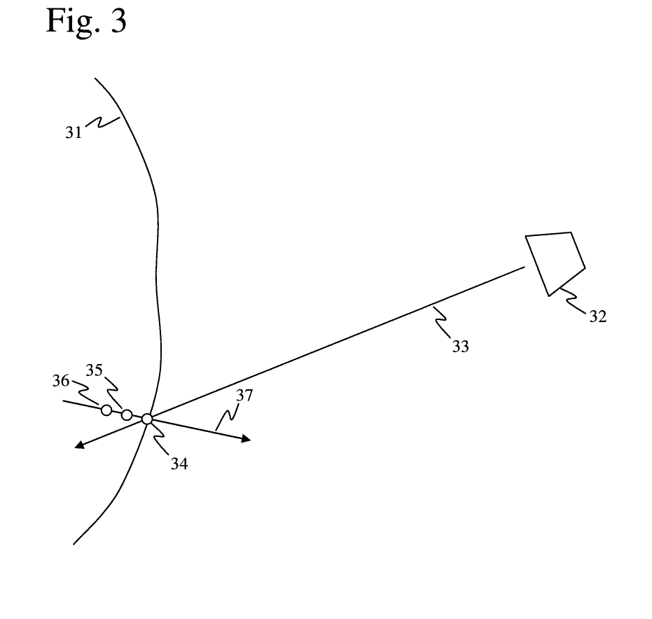

In addition to continuously recalculating surface shading, exemplary embodiments of the present invention utilize a novel approach to computing surface shading for mesh polygons that may provide for shading that remains accurate regardless of point of view. FIG. 3 is a diagram illustrating an approach for surface shading according to an exemplary embodiment of the present invention. According to this approach, a ray 33 is still cast from the point of view 32. The ray 33 intercepts the image volume 31 at a first voxel 34 on the surface of the image volume 31. However, rather than computing the surface shading along the ray 33, exemplary embodiments of the present invention may compute it along a direction 37 normal to the surface of the image volume 31. Thus the surface shading may be calculated by utilizing color and transparency information for voxels 34, 35, and 36.

Depending on the quality of the image volume and on the tool used to perform segmentation, the cardiac surface may appear complex and/or noisy. This in turn may cause parts of the surface of the image volume to contain small concave pockets which may cause the normal directions of adjacent regions to intersect. This may result in misleading visualization result, for example, where a scar would be visualized in multiple locations on the surface. To minimize or avoid this phenomenon, exemplary embodiments of the present invention may perform an optional step of smoothing the normals of the mesh (Step S115). An exemplary smoothing technique according to an embodiment of an invention smoothes the normal of the polygons comprising the mesh using a low-pass filter that is iteratively applied to the mesh. An example of low-pass filter is the mean filter. Use of the mean filter method may include iterating the vertex of the mesh, computing the mean of the normal of the neighborhood vertices and putting this value on the current vertex, although other techniques for normal smoothing may be used in addition to or in place of the mean filter or other low-pass filters. Rendering of the three-dimensional mesh, including the process of shading, may be performed, for example, using a ray tracing algorithm to perform a classic integration over the view direction. Alternatively, however, exemplary embodiments of the present invention may utilize a two-pass approach to rendering (Step S116). In the first pass, the mesh may be rendered in a depth buffer, for example, using a rasterization algorithm (Step S116a). This step may be implemented, for example, in a graphics processing unit (GPU) using an available hardware accelerated API such as OpenGL, DirectX, or GLSL.

In the second pass, a ray trace may be performed for each voxel (Step S116b). The ray trace may begin at the position of the surface of the heart using the depth buffer information and the camera configuration, an operation that is known as unprojection, and analysis may be performed for the volume following the smoothed normal of the surface for a certain distance that can be fixed or computed on-the-fly using smart algorithms. The result of the analysis may then be stored in the display buffer. Accordingly, the on-screen rendering, which may be performed quickly by the GPU, may be used to determine the depth of each polygon of the surface mesh and the calculating of depth from each voxel of the original image volume may be avoided.

The rendering is accordingly the result of the surface shading described above. After rendering has been performed, additional steps such as merging different information together, for example, classic surface rendering and the result of the Sobel filter of the scar rendering, may be performed to highlight the boundaries of the scars on the surface. Such subsequent steps may be implemented using image merging techniques.

This two-pass rendering approach (Step S116) may be used to automatically consider only the relevant volume data close to the cardiac surface rather than volume data that is above or far from the surface. Moreover, this approach may be more efficient and effective than classic ray tracing because it does analysis only where the segmented surface is visible thereby avoiding non-useful computation.

This added efficiency may allow for on-the-fly re-rendering as display parameters are changed and/or as viewing angle changes. Thus re-computation of surface shading may be performed in real-time.

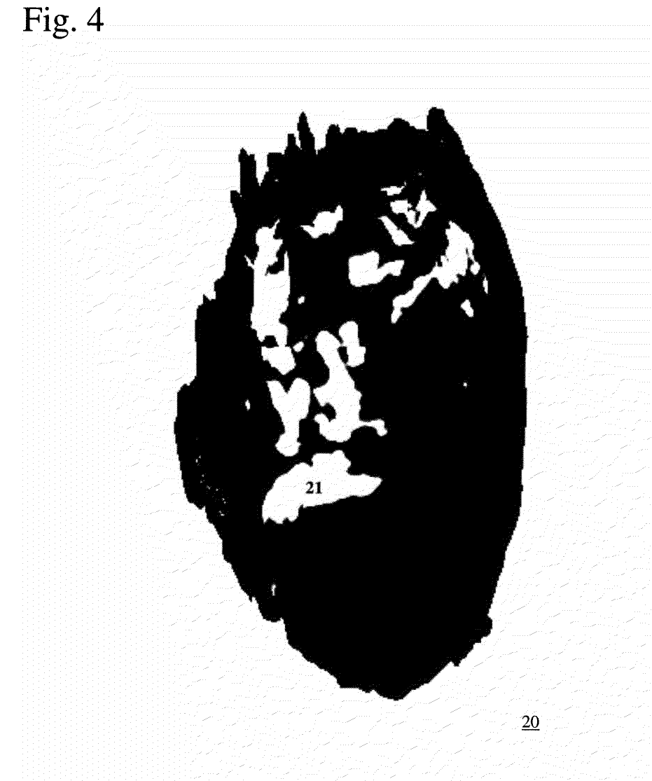

Once the mesh has been rendered, an example of which may be seen in FIG. 4, shading methods may be used to highlight the outline of scarring to improve visualization (Step S117). Examples of suitable methods for shading include Blinn-Phong shading using the classic normals of the mesh rather than the smoothed normals, etc.

The approach for two-pass rendering described above may also permit the avoidance of deformation when computing a texture for a surface by avoiding an unfolding step that may otherwise be necessary for algorithms that generate a texture that covers the whole surface.

In addition to or instead of highlighting the scar surface for enhanced viewing, exemplary embodiments of the present invention may automatically segment the scar surface (Step S118). Segmentation of the scar surface may include the performance of ray analysis to analyze the image volume over the normals of each surface mesh polygon so that the scar may be more easily segmented. When analyzing the volume over the normals, multiple techniques may be used. Examples of suitable techniques include maximum intensity projection (MIP), minimum intensity projection (MINIP), mean integration projections, combination of the above and/or the use of one or more other smarter filters.

Ray analysis may thus include the use of one or more filters. For example, one filter, according to an exemplary embodiment of the present invention, may involve computing along each ray both the mean and the maximum and then visualizing the difference between the mean and the maximum of the rays. The visualization may involve a threshold that has been found automatically or a global threshold provided by the user that can segment the scars. FIG. 4 is an example of a real-time segmentation result 20 that uses difference between max and mean along the ray to visualize cardiac surface scarring (white shapes, an example of which is referenced as 21) according to an exemplary embodiment of the present invention.

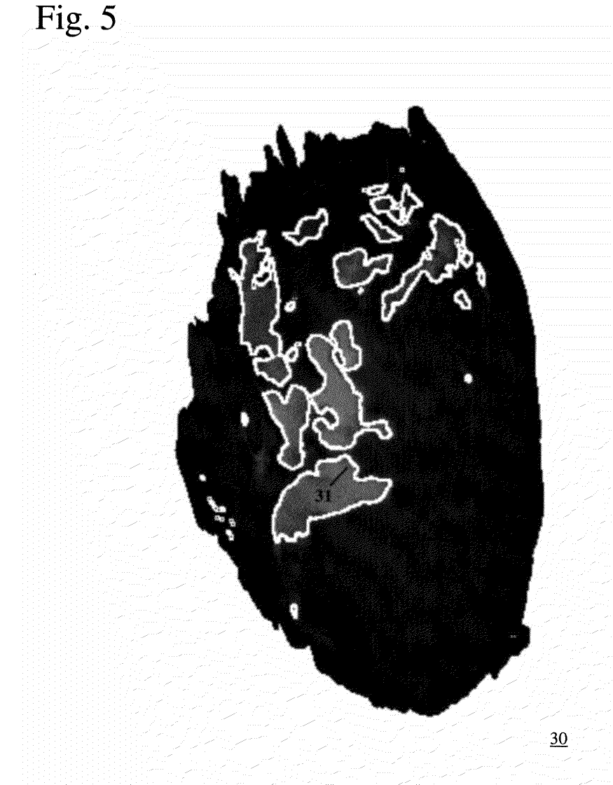

Highlighting and segmentation of the scar surface may be considered post-processing. Post-processing may be included in an optional embodiment of the present invention. According to one exemplary embodiment of the present invention, segmentation of the scar surface (Step S118) may occur after the highlighting (Step S117). In such a case, the highlighting results may be used to facilitate segmentation. For example, the derivative of the segmentation results may be calculated to highlight the contours of the scar segmentation. Derivatives may be calculated, for example, using the Sobel filter discussed above. It may also be possible to combine such visualization with rendering of a classic MIP. FIG. 5 is an example of a real-time segmentation result 30 including scar highlighting (white outlines, an example of which is referenced as 31) using derivative of the segmented image according to an exemplary embodiment of the present invention. This derivative-based highlighting may thus be used to better highlight the scars while showing the classical rendering in areas that do not have the scar. The implementation of this method and its varieties can be performed in real time, for example, on a GPU using an available hardware accelerated API such as OpenGL or DirectX using for example API such as OpenGL or DirectX.

After rendering has been performed, exemplary embodiments of the present invention may be efficient enough to provide for re-rendering to refresh the image, for example, 20 to 60 times per second or more. Accordingly, rendering may be performed in real-time. Exemplary embodiments of the present invention may also permit a user to change parameters to fine tune, in real-time, the rendering and scar highlighting/segmentation (Step S119).

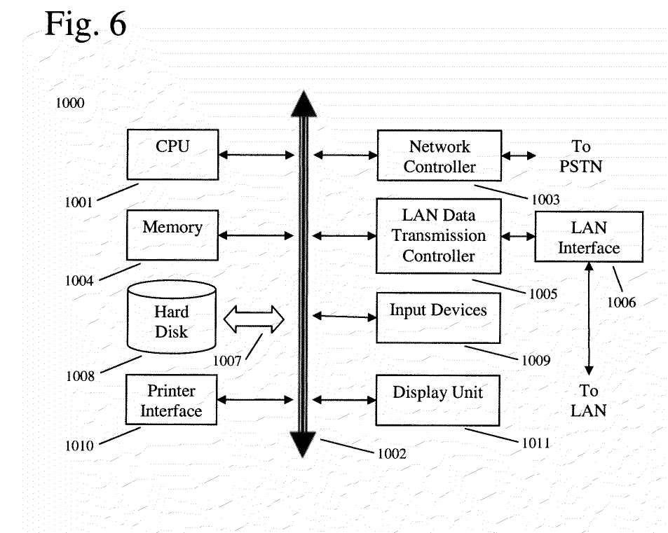

FIG. 6 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007.

Exemplary embodiments described herein are illustrative, and many variations can be introduced without departing from the spirit of the disclosure or from the scope of the appended claims. For example, elements and/or features of different exemplary embodiments may be combined with each other and/or substituted for each other within the scope of this disclosure and appended claims.

What is claimed is:

1. A method for imaging a myocardial surface, comprising:
receiving an image volume;
segmenting a myocardial surface within the received image volume;
extracting a polygon mesh of the segmented myocardial surface;
calculating a surface texture from voxel information taken along a path normal to the surface of the myocardium; and
rendering a view of the myocardial surface, said rendering including imposing the calculated surface texture onto the polygon mesh.

2. The method of claim 1, wherein the image volume is received from an image database, a computed tomography (CT) scanner, or a C-arm CT scanner.

3. The method of claim 1, wherein segmentation of the myocardial surface includes loading a pre-determined segmentation or calculating segmentation by applying a detection algorithm to the image volume.

4. The method of claim 1, wherein extracting a polygon mesh is performed by applying a marching squares approach to the segmented myocardial surface.

5. The method of claim 1, wherein rendering the view of the myocardial surface includes rendering the polygon mesh in a depth buffer of a graphical processing unit (GPU) using a rasterization algorithm.

6. The method of claim 5, wherein position information for the myocardial surface is extracted from the rendering of the myocardial surface rather than from the image volume.

7. The method of claim 5, wherein calculating the surface texture from voxel information taken along the path normal to the surface of the myocardium is performed from the rendering data stored in the depth buffer and camera settings.

8. The method of claim 1, wherein scarring is automatically segmented from the rendered surface mesh.

9. The method of claim 8, further including highlighting regions of scarring on the rendering of the myocardial surface.

10. The method of claim 9, wherein highlighting of scarring includes calculating a derivative of the segmentation of the scarring.

11. The method of claim 9, wherein the segmentation of the scarring are computed using ray analysis to analyze the image volume over the normals of each surface mesh polygon, wherein when analyzing the volume over the normals, smart filters, maximum intensity projection (MIP), minimum intensity projection (MINIP), mean integration projections, or a combination of the above may be used.

12. The method of claim 9, wherein highlighting of scarring includes application of a Sobel filter to the segmented regions of scarring.

13. The method of claim 1, additionally including allowing a user to change one or more parameters of display or segmentation and then re-rendering the view of the myocardial surface in real-time based on the changed parameters.

14. A method for applying texture to a polygon mesh, comprising:
casting a ray from a point of view, said ray intercepting a three-dimensional structure within an image volume;
determining a direction normal to the surface of the three-dimensional structure at the point at which the ray intercepts the surface of the structure;
analyzing a set of voxels of the three-dimensional structure along the normal direction including ascertaining voxel color and transparency;
combining the set of voxels based on the ascertained color and transparency to create a texture element; and
applying the created texture element to the surface.

15. The method of claim 14, wherein rasterization is performed along the ray to find intersections of the ray and the myocardium

16. The method of claim 14, wherein prior to determining the direction normal to the surface of the structure, normals of the surface of the structure are smoothed.

17. The method of claim 14, wherein the three-dimensional structure includes a myocardium and the polygon mesh is a representation of a surface of the myocardium.

18. A computer system comprising:
a processor; and
a non-transitory, tangible, program storage medium, readable by the computer system, embodying a program of instructions executable by the processor to perform method steps for imaging a myocardial surface, the method comprising:
receiving an image volume;
segmenting a myocardial surface within the received image volume;
extracting a polygon mesh of the segmented myocardial surface;
calculating a surface texture from voxel information taken along a path normal to the surface of the myocardium;
rendering a view of the myocardial surface, said rendering including imposing the calculated surface texture;
segmenting the scarring on the rendering of the myocardial surface; and
highlighting the scarring on the rendering of the myocardial surface based on the segmentation.

19. The computer system of claim 18, wherein rendering the view of the myocardial surface includes rendering the polygon mesh in a depth buffer of a graphical processing unit (GPU) using a rasterization algorithm.

20. The computer system of claim 19, wherein calculating the surface texture from voxel information taken along the path normal to the surface of the myocardium is performed from the rendering data stored in the depth buffer.

21. The computer system of claim 19, wherein position information for the myocardial surface is extracted from the rendering of the myocardial surface rather than from the segmented image volume.
